(19) United States
(12) Patent Application Publication
(10) Pub. No.: US 2024/0325374 A1
(43) Pub. Date: Oct. 3, 2024

(54) INHIBITING INTRACELLULAR PATHOGENS

(71) Applicant: THE RESEARCH INSTITUTE AT NATIONWIDE CHILDREN'S HOSPITAL, Columbus, OH (US)

(72) Inventors: Ky Van Hoang, Hilliard, OH (US); John S. Gunn, Powell, OH (US); Christopher Melander, Notre Dame, IN (US); Amy Sorge, South Bend, IN (US); Katherine Woolard, Notre Dame, IN (US); Morgan Carpenter, Notre Dame, IN (US)

(21) Appl. No.: 18/290,772

(22) PCT Filed: Jul. 25, 2022
(86) PCT No.: PCT/US2022/038183

§ 371 Date: Jan. 20, 2024

Related U.S. Application Data
(60) Provisional application No. 63/225,082, filed on Jul. 23, 2021.

(51) Int. Cl. A61K 31/4704; A61K 31/407; A61P 31/04

(52) U.S. Cl. CPC A61K 31/4704; A61K 31/407; A61P 31/04

(57) ABSTRACT

A method of treating or preventing infection by an intracellular pathogen in a subject is described. The method includes administering to the subject a therapeutically effective amount of a composition including KH-1, KH-2, or a derivative and/or a pharmaceutically acceptable salt thereof. A method of treating or preventing bacterial inflammation in a subject is also described. New KH-1 and KH-2 derivatives are also described.

### CROSS-REFERENCE TO RELATED APPLICATIONS

This application is a 371 of PCT International Patent Application No. PCT/US2022/038183, filed Jul. 25, 2022, which claims priority to U.S. Provisional Application Ser. No. 63/225,082, filed on Jul. 23, 2021. All the referenced applications which are hereby incorporated by reference in its entirety.

### BACKGROUND

Salmonella species (spp.) are Gram-negative facultative intracellular bacterial pathogens responsible for approximately 1.3 billion human infections annually worldwide. Salmonella infection results in two primary clinical manifestations: gastroenteritis and typhoid fever. Gastroenteritis is caused by non-typhoidal Salmonella serovars that are the most common cause of death from diarrheal disease and are the leading cause of foodborne disease outbreaks in the United States. Typhoid fever is caused by Salmonella enterica serovar Typhi (S. typhi) and various Salmonella Paratyphi pathovars. It is a systemic disease that leads to altered mental states, ileus, gastrointestinal bleeding, intestinal perforation, septic shock, and death. There are more than 14 million cases of typhoid fever and 136,000 deaths per year worldwide. S. typhi is a human-restricted serovar and is unable to colonize in mice; however, the related serovar S. typhimurium causes a typhoid fever-like disease in mice and is used as a model to study human typhoid fever as it recapitulates many characteristics of the human disease.

Upon infection through ingestion of contaminated food and/or water, Salmonella spp reach the lower gastrointestinal tract, traverse the intestinal epithelium through the M cells and enter the lamina propria and lymphoid follicles where bacteria are taken up by macrophages and reside in Salmonella-containing vacuoles (SCVs). In Typhoidal infection, the Salmonella-containing macrophages disseminate bacteria to common distal sites of the body, including the spleen, liver, and bone marrow. Dougan, G., et al., Immunol Rev, 2011. 240(1): p. 196-210 Virulence factors critical for the induction of proinflammatory responses in infected macrophages include pathogen-associated motifs (e.g., LPS, flagellin) that stimulate innate immunity and pro-inflammatory effectors. Inside SCVs, the bacteria utilize a type III secretion system to deliver many protein effectors into the cytoplasm to modulate the host immune system and alter vesicle trafficking, benefiting bacterial replication and dissemination. Salmonella infection robustly induces proinflammatory cytokines including tumor necrosis factor (TNFα), IL-6, and triggers caspase-1 dependent proinflammatory programmed cell death. The induction of the inflammatory response and bacterial-associated cell death is a bacterial strategy to promote disease.

The emergence of multi-drug resistance is increasingly recognized among S. typhi lineages and is a leading cause of treatment failure. Britto, C. D., et al., PLoS Negl Trop Dis, 2018. 12(10): p. e0006779. The current treatments for typhoid fever typically rely on antibiotics that directly target Salmonella (e.g., protein biosynthesis inhibition, membrane disruption, or DNA/RNA synthesis inhibition) and therefore have the highest and most direct efficacy when the bacterium is growing in the extracellular environment. Once the bacterium resides intracellularly, however, it adds a level of complexity to treatment options. The S. typhi multidrug-resistant strains overcome the first-line drugs for therapy, including ampicillin, chloramphenicol, and trimethoprim/sulfamethoxazole. Zaki, S. A. and S. Karande, J Infect Dev Ctries, 2011. 5(5): p. 324-37. Epidemiological studies in Pakistan and India showed that 73.7% (n=80), 56.2%, and 52.5% of S. typhi isolates had developed resistance to sulfonamide, ampicillin, or streptomycin, respectively with 58.7% of the overall isolates having multi-drug resistance. Afzal, A., et al., J Infect Dev Ctries, 2013. 7(12): p. 929-40. Vaccine approaches provide variable protection from Salmonella infections and can cause adverse side effects. The development of canonical antimicrobials that directly target S. typhi must continue, but additional approaches are also urgently needed, including host-targeted therapy.

Host-targeted therapy can interfere with host immune pathways that are required by a pathogen for productive replication and persistence. It may also enhance the immune response by stimulating host pathways that are involved in host defense against the pathogen or those that are perturbed and dysbalanced by a pathogen at the site of infection. These approaches can be used alone or in combination with traditional antibiotics. Not only does the host-directed therapeutic lessen the pathogen's ability to evade clearance by the immune system but also limits its development of resistance since the therapeutic is not directed at the pathogen itself. In fact, host-targeted therapy has been developed to treat infections by several intracellular bacterial pathogens (Johnson, M. M., et al., Mol Pharm, 2018. 15(11): p. 5336-5348), viral diseases (Moore, T. W., et al., ACS Med Chem Lett, 2013. 4(8): p. 762-767), and malaria (Achtman, A. H., et al., Sci Transl Med, 2012. 4(135): p. 135ra64).

### SUMMARY OF THE INVENTION

Host-targeted therapeutics present a promising anti-infective strategy against intracellular bacterial pathogens. A cell-based assay identified a compound that inhibits Salmonella proliferation in infected cells, 2-(3-hydroxypropyl)-1-(3-phenoxyphenyl)-1,2-dihydrochromeno[2,3-c]pyrrole-3,9-dione (KH-1), which is devoid of direct activity against Salmonella. The compound inhibits the growth of both antibiotic sensitive and resistant Salmonella strains inside macrophages and reduces LDH release from Salmonella infected cells. Subsequent screening of KH-1 commercial analogs identified 2-(4-fluorobenzyl)-1-(3-phenoxyphenyl)-1,2-dihydrochromeno[2,3-c] pyrrole-3,9-dione (KH-1-2) that is more effective in controlling Salmonella growth inside macrophages. In vitro KH-1-2 treatment of Salmonella infection resulted in an 8- to 10-fold reduction in bacterial load in infected macrophages. In combination with suboptimal ciprofloxacin treatment, KH-1-2 further reduces Salmonella growth inside macrophages. The toxicity and efficacy of KH-1-2 in controlling Salmonella infection were examined in vivo using a mouse model of Typhoid fever. No significant compound-related clinical signs and histological findings of the liver, spleen, or kidney were observed from uninfected mice that were intraperitoneally treated with KH-1-2. KH-1-2 significantly protected mice from a lethal dose of infection by an antibiotic-resistant Salmonella strain.

### BRIEF DESCRIPTION OF THE FIGURES

FIG. 1 provides a schematic of the screen to identify the host-targeted compounds.

FIGS. 2A-2E providing a chemical structure and graphs relating to the identification of KH-1. (A) Chemical structure of KH-1. (B) KH-1 reduced LDH release from S. typhimurium-infected macrophages. J774.1 macrophages were infected with S. typhimurium and the infected cells were treated with various concentrations of KH-1. The control group was treated with DMSO (negative control) or gentamicin (positive control). LDH release was evaluated at 24 hours post-treatment. KH-1 inhibited the growth of wild-type S. typhimurium inside the infected macrophages. J774.1 (C) or THP-1 (D) macrophages were infected with wild-type S. typhimurium and the infected cells were treated with various concentrations of KH-1. KH-1 inhibited the growth of ciprofloxacin-resistant S. typhimurium. (E) J774.1 macrophages were infected with ciprofloxacin-resistant S. typhimurium then the infected cells were treated with various concentrations of KH-1. The intracellular bacterial growth in (C, D, and E) was determined at 24 hrs post-treatment. The data were presented as a representative of three independent experiments. NS; not significant., n=3, ***p<0.001.

FIGS. 3A-3C provide graphs showing (A) KH-1 does not target Salmonella directly. S. typhimurium was grown in Luria-Bertani (LB) broth supplemented with various concentrations of KH-1. The bacterial growth was examined at the indicated time points by measuring optical density at 600 nm (OD600) (n=3). KH-1 pre-treatment does not affect Salmonella virulence. S. typhimurium grown in LB with 20 μM KH-1 or DMSO vehicle was used to infect J774.1 macrophages. The intracellular bacteria were recovered at 0 (B) and 24 hours (C) post-infection. n=3, NS: not significant.

FIGS. 4A & 4B provide graphs showing KH-1 sensitizes bacteria to ciprofloxacin in the infected macrophages but not in a standard medium. (A) S. typhimurium-infected J774.1 macrophages were treated with various concentrations of KH-1 with or without sub-optimal doses of ciprofloxacin. Intracellular bacteria were recovered at 24 hours post-treatment. (B) S. typhimurium was grown in LB broth with various concentrations of KH-1 with or without a sub-optimal dose of ciprofloxacin (0.08 μg/ml). The bacterial growth was evaluated by measuring OD₆₀₀ at different time points. n=3, ***p<0.001.

FIGS. 5A-5D provide a chemical structure and graphs relating to the examination of KH-1-2. (A) Chemical structure of KH-1-2. KH-1-2 has enhanced anti-Salmonella activity. (B) S. Typhi infected J774.1 macrophages were treated with various concentrations of KH-1 or KH-1-2. Intracellular bacteria were recovered at 24 hrs post treatment. (C) S. typhimurium infected J774.1 cells were treated with different concentrations of KH-1 or KH-1-2 or control DMSO. Intracellular bacteria were recovered at 24 hrs post treatment. The reduction in bacterial growth was calculated as percentage of CFUs recovered from treated group to the DMSO control. (D) KH-1-2 does not induce ROS production in neutrophils. PLB-985 neutrophil-like cells were treated with different concentrations of KH-1-2. The ROS production was monitored at different time points. The positive control group was treated with 0.1 μg PMA/ml. The data presented in B, C, and D are representative of at least three independent experiments. n=3, ***p<0.001.

FIG. 6 provides an image showing the histopathological studies of organs from mice treated with KH-1-2 for 10 consecutive days via I.P route (20× magnification). At the end of the experiment, mice were sacrificed, and liver, spleen, and kidney were collected, fixed in 4% paraformaldehyde for 72 hrs, processed and stained with hematoxylin and eosin (H&E). No increase in immune cell migration and no significant apoptosis was observed in the spleen and kidney between the control and KH-1-2 treatment groups. For the liver histopathology studies, KH-1-2 treatment at 1 mg resulted in no adverse effects while treatment at 10 mg resulted in minimal effects indicated by infiltration of lymphocytic cells and neutrophils.

FIG. 7 provides a graph showing KH-1-2 treatment protects mice from lethal S. typhimurium infection. Mice (4 or 5 mice per group) were orally infected with a lethal dose (10⁶ CFU/mouse) of ciprofloxacin-resistant S. typhimurium. One day post-infection, the infected mice were given KH-1-2 prepared in 200 μl PBS at 0.05, 0.1, and 0.25 mg/kg per day via the intraperitoneal route for 14 consecutive days. The infected mice were monitored for survival for up to 2 weeks post-infection. DPI: days post-infection, *p<0.05 with respect to PBS control.

FIG. 8 provides a synthetic scheme for the KH-1-2 derivative.

FIG. 9 provides a graph showing KH-2 reduced lactate dehydrogenase (LDH) release from Salmonella infected J774.1 macrophages.

FIG. 10 provides a graph showing KH-2 inhibits the growth of Salmonella in J774.1 macrophages.

FIG. 11 provides a graph showing that KH-2 does not inhibit the growth of Salmonella in Luria-Bertani (LB) bacterial growth media.

FIG. 12 provides a graph showing that KH-2 protects mice from lethal Salmonella infection.

### DETAILED DESCRIPTION OF THE INVENTION

The inventors have demonstrated a method of treating or preventing infection by an intracellular pathogen in a subject, comprising administering a therapeutically effective amount of a composition including KH-1, KH-2, or a derivative thereof, and/or a pharmaceutically acceptable salt thereof. The inventors have also demonstrated a method of inhibiting bacterial inflammation, and novel KH-1 and KH-2 derivatives have been identified.

### Definitions

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

As used herein, “a subject in need” refers to a subject who has, or has an increased risk for developing an infection by an intracellular pathogen, an increased susceptibility to infection by an intracellular pathogen, or an increased susceptibility to developing bacterial inflammation. A subject may have an increased risk due to being immunosuppressed or having been exposed to a bacterial pathogen, for example.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for KH-1 and KH-2 derivatives are those that do not interfere with the compound's ability to inhibit the growth of intracellular pathogens. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Lower alkyl groups are those including at most 6 carbon atoms. Examples of alkyl groups include haloalkyl groups and hydroxyalkyl groups. Cyclic groups can be monocyclic or polycyclic and preferably have from 3 to 10 ring carbon atoms.

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. A halo moiety can be chlorine, bromine, fluorine, or iodine.

The term “aryl” as used herein includes carbocyclic aromatic rings or ring systems. Examples of aryl groups include phenyl, naphthyl, biphenyl, anthracenyl, phenanthracenyl, fluorenyl and indenyl. Aryl groups may be substituted or unsubstituted.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The terms “arylene” and “heteroarylene” are the divalent forms of the “aryl” and “heteroaryl” groups defined above. The terms “arylenyl” and “heteroarylenyl” are used when “arylene” and “heteroarylene”, respectively, are substituted. For example, an alkylarylenyl group comprises an arylene moiety to which an alkyl group is attached.

When a group is present more than once in any formula or scheme described herein, each group (or substituent) is independently selected, whether explicitly stated or not. For example, for the formula —C(O)—NR₂ each R group is independently selected.

As a means of simplifying the discussion and the recitation of certain terminology used throughout this application, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

“Pharmaceutically acceptable” as used herein means that the compound or composition is suitable for administration to a subject to achieve the treatments described herein, without unduly deleterious side effects in light of the severity of the disease and necessity of the treatment.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, etc.

Prevention, as used herein, refers to therapy providing a benefit to a subject having an increased risk of being afflicted with a condition or disease such as infection, including avoidance of or a decrease of one or more symptoms of the disease should the disease occur.

### Treating or Preventing Infection by an Intracellular Pathogen

In one aspect, the present invention provides a method of treating or preventing infection by an intracellular pathogen in a subject. The method includes administering a therapeutically effective amount of a composition including KH-1, KH-2, or a derivative thereof and/or a pharmaceutically acceptable salt thereof. The structures of KH-1 and KH-2 are shown below:

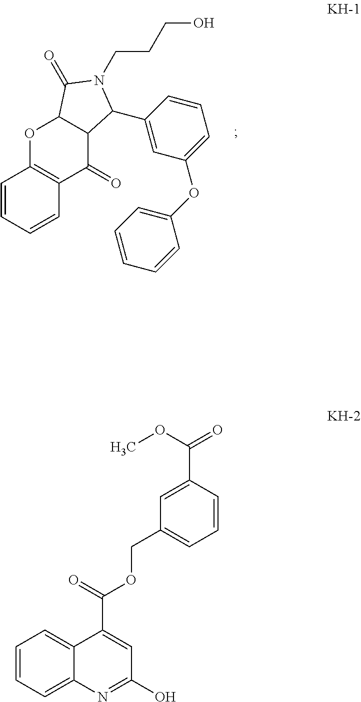 [SMILES: O=C1c2ccccc2OC2C(=O)N(CCCO)C(c3cccc(Oc4ccccc4)c3)C12 ; COC(=O)c1cccc(COC(=O)c2cc(O)nc3ccccc23)c1]

A KH-1 or KH-2 derivative is a compound substantially similar to KH-1 or KH-2, or a compound that can be readily imagined to arise from KH-1 or KH-2 if a group is attached to the compound or an atom or group is replace with another atom or group. KH-1 or KH-2 derivatives also include structural analogs of KH-1 or KH-2 in which a small number of atoms in the structural backbone have been replaced with a similar atom, such as replacement of an oxygen or carbon atom with a nitrogen atom. A number of specific KH-1 and KH-2 derivatives are described herein.

In some embodiments, the compound is KH-1 (2-(3-hydroxypropyl)-1-(3-phenoxyphenyl)-1,2-dihydrochromeno[2,3-c]pyrrole-3,9-dione) or a pharmaceutically acceptable salt thereof. In other embodiments the compound is KH-2 (3-(methoxycarbonyl)benzyl 2-hydroxy-4-quinolinecarboxylate) or a pharmaceutically acceptable salt thereof. In a further embodiment, the compound is KH-1-2 (2-(4-fluorobenzyl)-1-(3-phenoxyphenyl)-1,2-dihydrochromano[2,3-c]pyrrole-3,9-dione or a pharmaceutically acceptable salt thereof.

In some embodiments a method of treating or preventing infection by an intracellular pathogen in a subject is provided, in which the method includes administering to the subject a therapeutically effective amount of a compound according to Formula I:

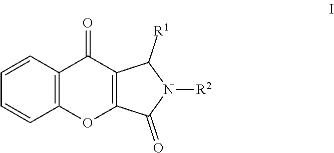

wherein R¹ is a C₆-C₁₂ alkyl, phenyl, or heteroaryl group, and R² is C₁-C₆ alkyl group or a phenyl group, wherein one or more of the hydrogens of the phenyl group are optionally substituted with a methyl, halogen, —OMe, or —OPh moiety, or a pharmaceutically acceptable salt thereof.

Various additional embodiments for the use of compounds of Formula I are contemplated. In some embodiments, R¹ is a heteroaryl group, while in further embodiments R¹ is a diphenyl ether. In further embodiments, R¹ is a diphenyl ether and R² is n-propanol (i.e., —CH₂CH₂CH₂OH), providing the compound also known as KH-1. In further embodiments, R² a phenyl group, wherein one or more of the hydrogens of the phenyl group are substituted with a halogen.

Alternately, or in addition, in some embodiments, a method of treating or preventing infection by an intracellular pathogen in a subject is provided, in which the method includes administering to the subject a therapeutically effective amount of a compound according to Formula II:

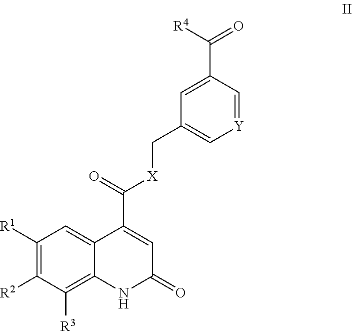

wherein R¹, R², and R³ are either a hydrogen or halogen, wherein R⁴ is —NHMe or —OMe, X is O or NH, and Y is C or N; or a pharmaceutically acceptable salt thereof. In some embodiments, a compound in which R¹-R³ are —H, R⁴ is —OMe, X is O, and Y is C, which corresponds to the compound KH-2.

The method of treatment or prevention can also include any of the specific KH-1 and/or KH-2 derivatives described further herein, such as compound 2-039, 2-017, 2-043, 2-047, 2-0129, 2-041, 2-027, 2-037, 2-052, 2-015, 2-025, 2-055, 2-031, MC-1-9. MC-1-17, MC-1-9, MC-1-17, MC-1-26, MC-1-40, MC-1-41, AKS-1-001, AKS-1-002, AKS-1-003, AKS-1-004, AKS-1-005, AKS-1-006, and AKS-1-007.

The present invention encompasses both prophylactic (preventive) and therapeutic treatment. In some embodiments, a method of preventing infection by an intracellular pathogen is provided. KH-1 or KH-2 (or their derivatives; e.g., KH-1-2) can, for example, be administered prophylactically to a mammal prior to exposure to infection by an intracellular pathogen. Prophylactic administration, also referred to as prevention, is effective to decrease the likelihood of the subsequent infection in the mammal, or to decrease the severity of infection that subsequently occurs.

Alternatively, or in addition, a method of treating infection by an intracellular pathogen is provided. KH-1 or KH-2 (or their derivatives; e.g., KH-1-2) can, for example, be administered therapeutically to a subject that is already infected. In one embodiment of therapeutic administration, administration of the compound(s) effective to eliminate the infection; in another embodiment, administration of the compound(s) is effective to decrease the severity of the infection. The subject is preferably a mammal, such as a domesticated farm animal (e.g., cow, horse, pig) or pet (e.g., dog, cat). More preferably, the subject is a human.

Intracellular pathogen are pathogenic microorganisms (e.g., bacteria) that are able to invade or be engulfed by cells and survive within a membrane-bound compartment. See Ray et al., Nat Rev Microbiol., 7(5):333-40 (2009). Examples of intracellular pathogens, and their associated diseases, including, but are not limited to, Mycobacterium tuberculosis (tuberculosis), Francisella spp. (pulmonary tularemia), Listeria monocytogenes, Shigella flexneri, Burkholderia pseudomallei, Group A Streptococcus pyogenes, Rickettsiae spp., Chlamydia spp., and Salmonella spp.

In some embodiments, the intracellular pathogen is a Francisella species. In further embodiments the Francisella species is Francisella tularensis. Francisella tularensis is a gram-negative, facultative, highly virulent bacterium, which causes the zoonotic disease tularemia. Infection can occur through several routes, but pneumonic tularemia is the most severe clinical form, with a mortality rate up to 60 percent in the absence of treatment. F. tularensis can invade a range of host cells, but its primary target in vivo is the macrophage. Sjostedt, A, Curr. Opin. Microbiol. 6, p. 66-71 (2003). After being phagocytosed by macrophages, this intracellular pathogen can block the fusion of Francisella-containing phagosomes with lysosomes and escape from the phagosome into the cytosol where it multiplies. Following proliferation within macrophages, F. tularensis induces host cell apoptosis or pyroptosis leading to the release of bacteria and subsequent infection of new cells. Francisella tularensis includes the subspecies tularensis (type A), palearctica (type B), novicida, and mediasiatica.

In some embodiments, the intracellular pathogen is a Salmonella species. Salmonella represents a group of Gram-negative facultative anaerobic pathogenic bacteria which costs millions of lives across the world every year. The intracellular life-cycle of Salmonella includes the entry of the bacterium in the host cell, formation of a Salmonella containing vacuole (SCV), evasion of host immune response within the SCV, and ultimately host cell death by apoptosis. See Pucciarelli, M G. and Garcia-Del Protillo F., Microbiol Spectr., 5(4) (2017). In some embodiments, the Salmonella species is Salmonella typhimurium. while in other embodiments the Salmonella species is Salmonella typhi. While S. typhi infection is strictly limited to humans and higher primates, S. typhimurium has a wide range of host such as rodents, cattle and mammals.

In some embodiments, the intracellular pathogen inhabits macrophage cells. Macrophage cells, as used herein, refers to immune cells of the innate immune system, and include macrophages, macrophage-like cells, and macrophage precursors such as monocytes. Macrophage-like cells include tingible body macrophages, dendritic cells, foam cells, and multinucleated giant cells. In some embodiments, the Francisella tularensis infection is inhibited in macrophage cells, which are the primary in vivo target for F. tularensis. In additional embodiments, the KH-1 or KH-2 (or their derivatives) are able to inhibit F. tularensis in macrophages without significant toxicity to other cells, and macrophage cells in particular.

In some embodiments, the intracellular pathogen is antibiotic resistant. Antibiotics, as defined herein, are bactericidal or bacteriostatic compounds already known in the art. Examples of known antibiotics include agents that target the bacterial cell wall, such as penicillins, cephalosporins, agents that target the cell membrane such as polymixins, agents that interfere with essential bacterial enzymes, such as quinolones and sulfonamides, and agents that that target protein synthesis such as the aminoglycosides, macrolides and tetracyclines. Additional known antibiotics include cyclic lipopeptides, glycylcyclines, and oxazolidinones. Antibiotic resistance represents the ability of intracellular pathogens to decrease (i.e., resist) the cytotoxic and cytostatic effects of antibiotics.

In some embodiments, the KH-1, KH-2, or derivative thereof is used together with another antibacterial agent to provide combinational therapy of the intracellular pathogen. The combination of agents can provide additive or synergistic effects. The KH-1, KH-2, or derivative thereof may be administered before, simultaneously, or after administration of an additional agent useful for treating infection with an intracellular pathogen. In some embodiments, the effects of the drugs overlap one another in time.

A variety of different antibacterial agents can be used in combination with KH-1, KH-2, and their derivatives. Examples include quinolones (e.g., fluoroquinolones) such as ciprofloxacin, ansamycins, macrolides, and tetracyclines such as tigecyclin, and more exotic therapies such as the use of antisense oligonucleotides.

### Treating or Preventing Bacterial Inflammation

Another aspect of the invention provides a method of treating or preventing bacterial inflammation in a subject. The method includes administering a therapeutically effective amount of a composition including KH-1, KH-2, or a derivative thereof and/or a pharmaceutically acceptable salt thereof.

In some embodiments, the invention provides a method of treating or preventing bacterial inflammation in a subject, comprising administering to the subject a therapeutically effective amount of a composition according to Formula I:

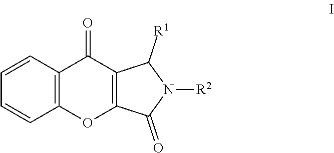

wherein R¹ is a C₆-C₁₂ alkyl, phenyl, or heteroaryl group, and R² is C₁-C₆ alkyl group or a phenyl group, wherein one or more of the hydrogens of the phenyl group are optionally substituted with a methyl, halogen, —OMe, or —OPh moiety; or Formula II:

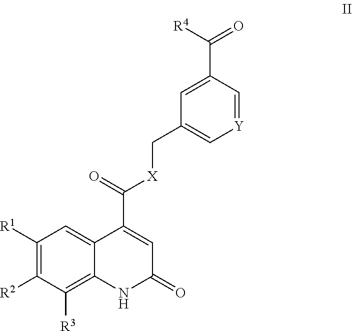

wherein R¹, R², and R³ are either a hydrogen or halogen, wherein R⁴ is —NHMe or —OMe, X is O or NH, and Y is C or N; or a pharmaceutically acceptable salt thereof.

In some embodiments, the compound is according to Formula I, while in other embodiments the compound is according to Formula II. The compound can include KH-1, KH-2, or any of their derivatives, as described herein.

Bacterial inflammation is inflammation in a subject caused by the presence of pathogenic bacteria. Pathogenic bacteria are harmful bacteria, typically as a result of their ability to cause an infection having harmful symptoms in a subject. Examples of pathogenic bacteria include Mycobacterium tuberculosis, Escherichia coli, Vibrio cholerae, Strepthococcus pneumoniae, and Staphylococcus aureus. Preferably, the pathogenic bacteria are those capable of inducing inflammation.

In some embodiments, the inflammation is systemic inflammation. Systemic inflammation is an exaggerated defense response of the body to a noxious stressor, such as infection, trauma, surgery, acute inflammation, ischemia or reperfusion, or malignancy, to localize and then eliminate the endogenous or exogenous source of the insult. Systemic inflammation caused by infection is also known as sepsis. Lipopolysaccharide (LPS) from pathogenic organisms such as intracellular pathogens can cause inflammation, including sepsis. See Calandra, T., J Chemother. Spec No 1(1):173-80 (2001).

### KH-1 and KH-2 Derivatives

The inventors have synthesized a number of new derivatives based on KH-1 and KH-2 that can be used according to the methods described herein. These include a number of new KH-1 derivatives according to Formula I:

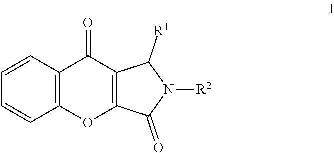

wherein R¹ is a C₆-C₁₂ alkyl, phenyl, or heteroaryl group, and R² is a phenyl group, wherein one or more of the hydrogens of the phenyl group are optionally substituted with a methyl, halogen, —OMe, or —OPh moiety, or a pharmaceutically acceptable salt thereof.

In some embodiments, R¹ is diphenyl ether, while in further embodiments one or more of the hydrogens of the phenyl group are substituted. For example, one or more of the hydrogen atoms may be substituted with a halogen. The KH-1 derivatives can be provided as part of a pharmaceutical composition including a pharmaceutically acceptable carrier. In some embodiments, the compounds can be biotinyulated.

Embodiments of the present invention provides KH-1 derivatives having the structures and designations shown below:

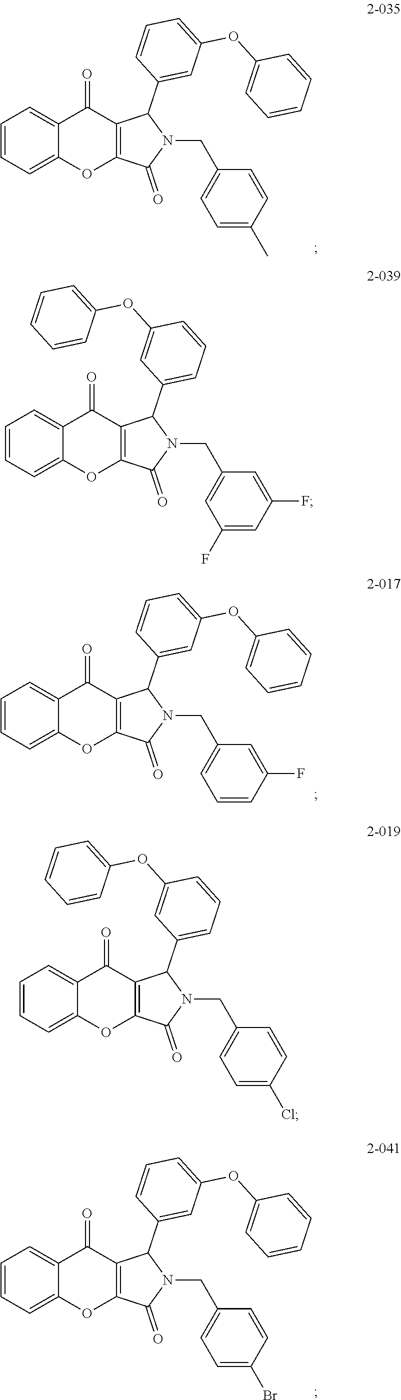

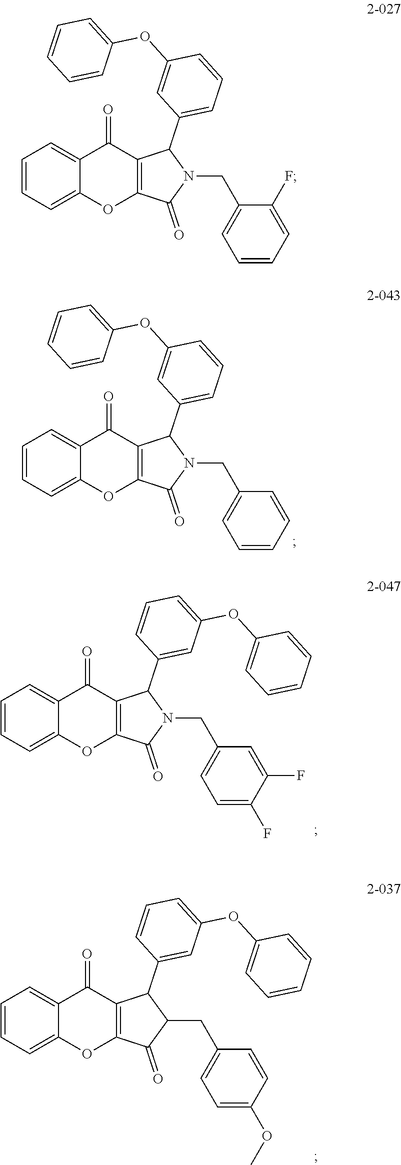 [SMILES: O=C1c2oc3ccccc3c(=O)c2C(c2cccc(Oc3ccccc3)c2)N1Cc1ccccc1F ; O=C1c2oc3ccccc3c(=O)c2C(c2cccc(Oc3ccccc3)c2)N1Cc1ccccc1 ; O=C1c2oc3ccccc3c(=O)c2C(c2cccc(Oc3ccccc3)c2)N1Cc1ccc(F)c(F)c1 ; COc1ccc(CC2C(=O)c3oc4ccccc4c(=O)c3C2c2cccc(Oc3ccccc3)c2)cc1]

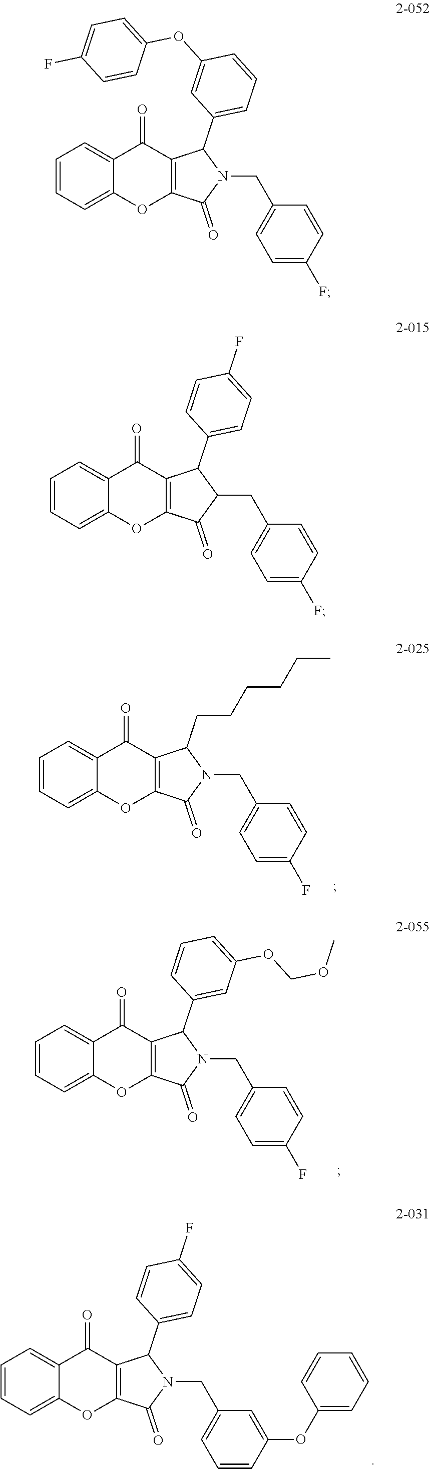 [SMILES: O=C1c2oc3ccccc3c(=O)c2C(c2cccc(Oc3ccc(F)cc3)c2)N1Cc1ccc(F)cc1 ; O=C1c2oc3ccccc3c(=O)c2C(c2ccc(F)cc2)C1Cc1ccc(F)cc1 ; CCCCCCC1c2c(oc3ccccc3c2=O)C(=O)N1Cc1ccc(F)cc1 ; COCOc1cccc(C2c3c(oc4ccccc4c3=O)C(=O)N2Cc2ccc(F)cc2)c1 ; O=C1c2oc3ccccc3c(=O)c2C(c2ccc(F)cc2)N1Cc1cccc(Oc2ccccc2)c1]

The inventors have also prepared a number of new KH-2 derivatives. Accordingly, another aspect of the invention provides a composition comprising a compound according to Formula II:

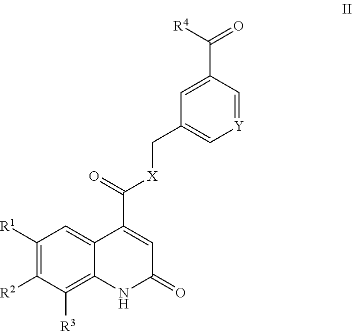

wherein R¹, R², and R³ are either a hydrogen or halogen, wherein R⁴ is —NHMe or —OMe, X is O or NH, and Y is C or N; or a pharmaceutically acceptable salt thereof.

In some embodiments, one or more of R¹, R², and R³ is a halogen. In further embodiments, X is NH, while in yet further embodiments R⁴ is —NHMe. The KH-2 derivatives can be provided as part of a pharmaceutical composition including a pharmaceutically acceptable carrier.

Embodiments of the present invention provide KH-2 derivatives having the structures and designations shown below:

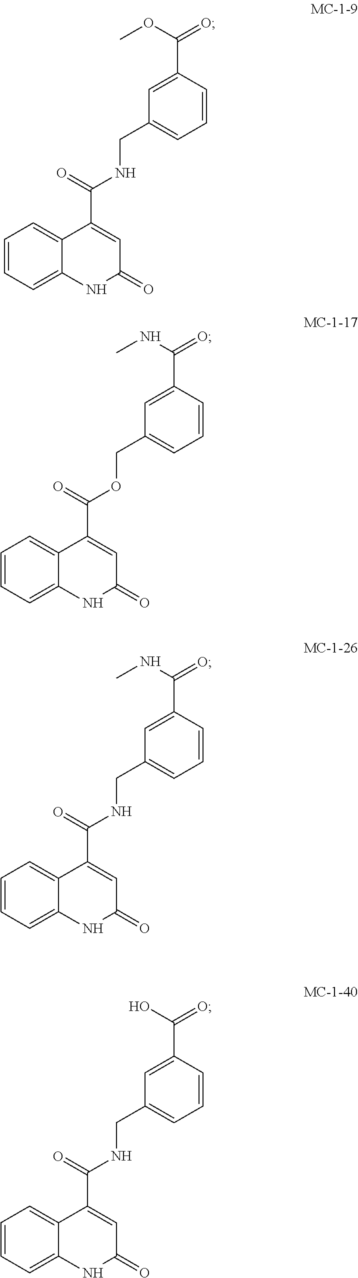 [SMILES: COC(=O)c1cccc(CNC(=O)c2cc(=O)[nH]c3ccccc23)c1 ; CNC(=O)c1cccc(COC(=O)c2cc(=O)[nH]c3ccccc23)c1 ; CNC(=O)c1cccc(CNC(=O)c2cc(=O)[nH]c3ccccc23)c1 ; O=C(O)c1cccc(CNC(=O)c2cc(=O)[nH]c3ccccc23)c1]

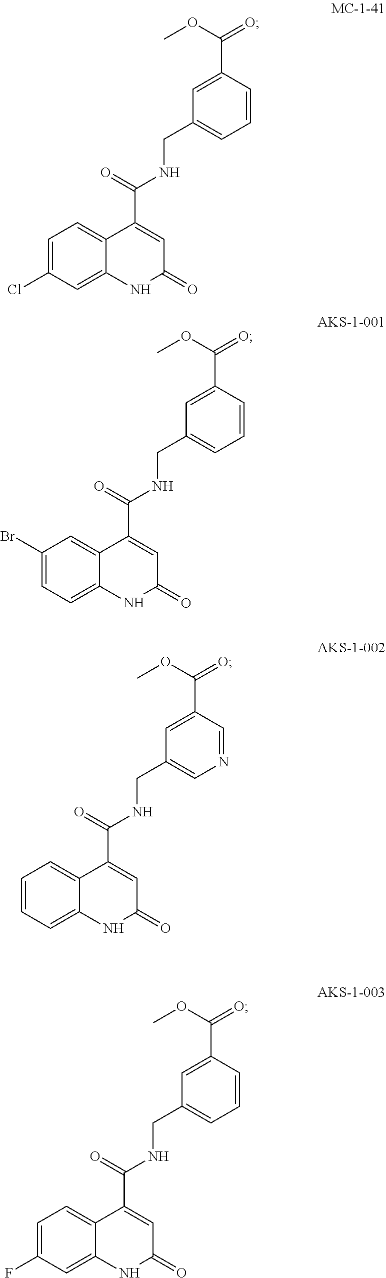 [SMILES: COC(=O)c1cccc(CNC(=O)c2cc(=O)[nH]c3cc(Cl)ccc23)c1 ; COC(=O)c1cccc(CNC(=O)c2cc(=O)[nH]c3ccc(Br)cc23)c1 ; COC(=O)c1cncc(CNC(=O)c2cc(=O)[nH]c3ccccc23)c1 ; COC(=O)c1cccc(CNC(=O)c2cc(=O)[nH]c3cc(F)ccc23)c1]

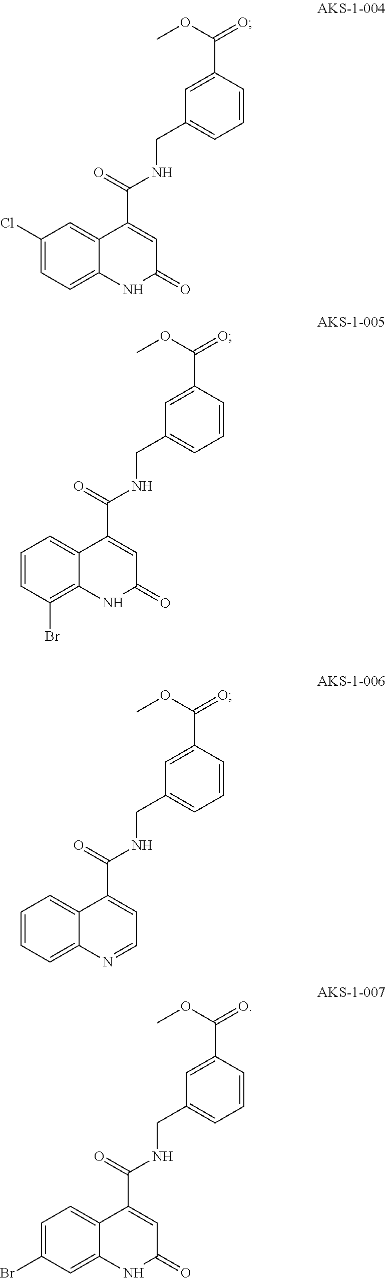 [SMILES: COC(=O)c1cccc(CNC(=O)c2cc(=O)[nH]c3ccc(Cl)cc23)c1 ; COC(=O)c1cccc(CNC(=O)c2cc(=O)[nH]c3c(Br)cccc23)c1 ; COC(=O)c1cccc(CNC(=O)c2ccnc3ccccc23)c1 ; COC(=O)c1cccc(CNC(=O)c2cc(=O)[nH]c3cc(Br)ccc23)c1]

### Administration and Formulation

The present invention also provides pharmaceutical compositions that include KH-1, KH-2, or their derivatives, as an active ingredient, and a pharmaceutically acceptable carrier or carriers, in combination with the active ingredient. Any of the compounds described above as being suitable for the treatment of intracellular pathogens can be included in pharmaceutical compositions of the invention.

The KH-1 or KH-2 (or their derivatives; e.g. KH-1-2) can be administered as pharmaceutically acceptable salts. Pharmaceutically acceptable salt refers to the relatively non-toxic, inorganic and organic acid addition salts of the KH-1, KH-2, or their derivatives. These salts can be prepared in situ during the final isolation and purification of KH-1 or KH-2, or by separately reacting purified KH-1, KH-2, or a derivative with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions include KH-1, KH-2, or their derivatives together with one or more of a variety of physiological acceptable carriers for delivery to a patient, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The KH-1, KH-2, or their derivatives can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The KH-1 or KH-2 (or their derivatives; e.g. KH-1-2) are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations of the present invention suitable for oral administration may be presented as discrete units such as tablets, troches, capsules, lozenges, wafers, or cachets, each containing a predetermined amount of the active agent as a powder or granules, as liposomes containing KH-1, KH-2, or their derivatives, or as a solution or suspension in an aqueous liquor or non-aqueous liquid such as a syrup, an elixir, an emulsion, or a draught. Such compositions and preparations typically contain at least about 0.1 wt-% of the active agent. The amount of KH-1, KH-2, or their derivatives (i.e., active agent) is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

### Preparation of the Compounds

Compounds of the invention may be synthesized by synthetic routes that include processes similar to those well known in the chemical arts, particularly in light of the description contained herein. The starting materials are generally available from commercial sources such as Aldrich Chemicals (Milwaukee, Wisconsin, USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

The present invention is illustrated by the following examples. It is to be understood that the particular examples, materials, amounts, and procedures are to be interpreted broadly in accordance with the scope and spirit of the invention as set forth herein.

### EXAMPLES

### Example 1: Identification of a Host-Targeted Compound to Control Typhoid Fever

The work described here details the development of a simple, cell-based high-throughput assay to screen potential host-targeted compounds to control Typhoid fever. Salmonella infection induces macrophage death indicated by the release of lactate dehydrogenase (LDH) in the medium. Using the LDH assay to measure cellular metabolic activity and cell viability, a subset of the ChemBridge eukaryotic kinase inhibitor/ATP mimetic library was screened for compounds that reduce Salmonella-associated cell death without direct antimicrobial activity against Salmonella in vitro. This study provides a viable lead candidate for the continuation of host-targeted drug discovery efforts with the goal of treatment of typhoid fever caused by both antibiotic-sensitive and resistant bacteria.

### Materials and Methods

### Cells and Bacterial Strains

J774.1 murine macrophages were cultured in Dulbecco modified Eagle medium (DMEM) (Gibco-Life Technologies, Grand Island, NY). THP-1 human macrophages and PLB-985 cells were cultured in RPMI 1640 (Gibco-Life Technologies, Grand Island, NY). Cells were maintained under humidified conditions at 37° C., 5% CO₂ in medium supplemented with 10% fetal bovine serum (FBS) (GIBCO-BRL) and penicillin-streptomycin (Gibco-Life Technologies, Grand Island, NY) (100 μg/ml each). The S. typhimurium wild-type strain ATCC 14028 and an S. typhi strain TY2 were used in this study. A clinical ciprofloxacin-resistant S. typhimurium isolate was collected from Ethiopia. Eguale, T., et al., Bmc Infectious Diseases, 2015. 15. All bacterial strains were cultured in Luria Bertani (LB) broth (Difco, Detroit, MI) and incubated at 37° C. with aeration.

### Compound Library, Reference Compounds, and Reagents

A 3,000-member ATP mimetic library in 96-well plate format was sourced from ChemBridge. The reference compounds 2-(3-hydroxypropyl)-1-(3-phenoxyphenyl)-1,2-dihydrochromeno[2,3-c] pyrrole-3,9-dione (KH-1), and 2-(4-fluorobenzyl)-1-(3-phenoxyphenyl)-1,2-dihydrochromeno[2,3-c] pyrrole-3,9-dione (KH-1-2) were purchased ChemBridge. KH-1-2 was resynthesized in house to confirm structure, purity, and activity. All commercial solvents and reagents were purchased from VWR or Sigma-Aldrich and used without any further purification. Reactions were monitored using thin layer chromatography (TLC) using glass-backed pre-coated silica gel plates from VWR (TLC Silica Gel 60 sheets, Millipore Sigma, F254, 60 Å pore 230-400 mesh) using UV visualization. Column chromatography was performed using silica gel (60 Å, particle size 40-60 m, VWR). Deuterated solvents for NMR characterization were purchased from Cambridge Analytical via VWR and used as-is. All NMR spectra were performed at room temperature and recorded on a Bruker AVANCE III HD 400 Nanobay spectrometer without the use of signal suppression function and calibrated using the residual undeuterated solvent peak (chloroform-d: δ 7.26 ppm ¹H NMR, 77.16 ppm 13C NMR). Proton (¹H) NMR are reported as follows: chemical shift in ppm (multiplicity, coupling constant(s) in Hz, relative integration). Abbreviations used are s, singlet; d, doublet; t, triplet; q, quartet; m, multiplet. High-resolution mass spectra (HRMS) were recorded on a Bruker micrOTOF II by electrospray ionization (ESI) time of flight (TOF) experiments using direct infusion in 9:1 acetonitrile: water. Analyses were performed by the Mass Spectrometry and Proteomics Facility at the University of Notre Dame and reported as m/z. KH-1-2 was characterized and tested at >95% purity as determined by liquid chromatography on a Bruker micrOTOF-Q II by the University of Notre Dame Mass Spectrometry and Proteomics Facility.

### Synthesis of KH-1-2

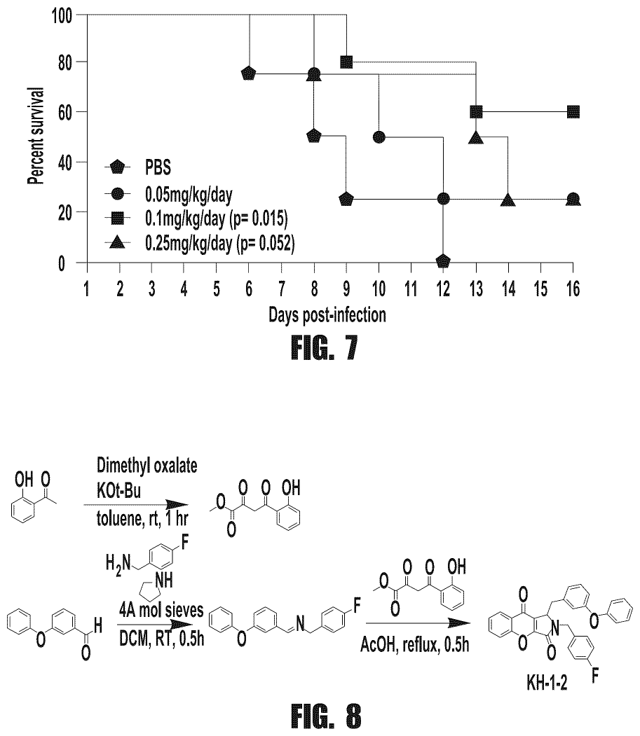

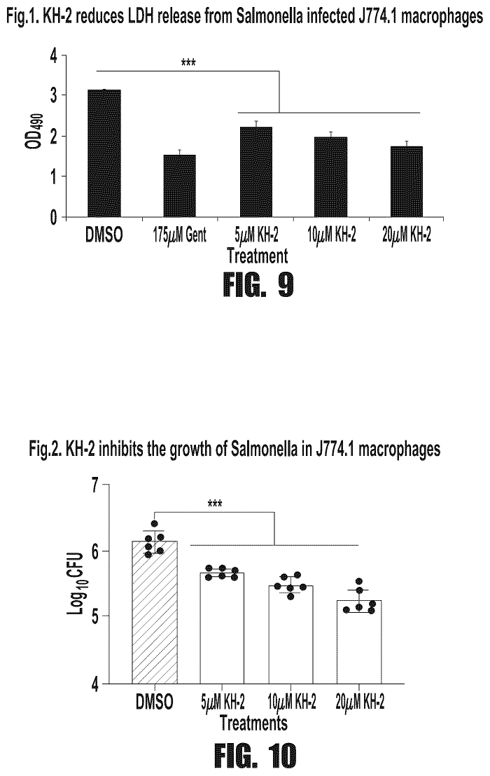

The KH-1-2 synthesis scheme is depicted in FIG. 8. Methyl 4-(2-hydroxyphenyl)-2,4-dioxobutanoate was synthesized as previously reported (US Patent Pub. 2009/0036451) from dimethyl oxalate and 2-hydroxyacetophenone. An oven-dried 10 mL round bottom flask was added 4-fluorobenzylamine (80 mg, 0.40 mmol, 1.0 equiv) and 4 Å molecular sieves in 1.25 mL dichloromethane. Anhydrous pyrrolidine (3.3 μL, 40.0 μmol, 0.10 equiv) was added directly to the reaction followed by 3-phenoxybenzaldehyde (60 mg, 0.48 mmol, 1.2 equiv). The reaction stirred for 30 minutes at room temperature and was monitored by TLC. The resulting solution was filtered, dried over anhydrous sodium sulfate, filtered, and evaporated in vacuo to yield (E)-N-(4-fluorobenzyl)-1-(3-phenoxyphenyl)methanimine. This crude oil was used directly in the next reaction without further purification.

Following the literature procedure for 1,2-diaryl-1,2-dihydro-chromeno[2,3-c]pyrrole-3,9-diones (Vydzhak, R. N., Simple synthesis of 1,2-diaryl-1,2-dihydro-chromeno[2,3-c]pyrrole-3,9-diones. Russ J Gen Chem 2006), methyl 4-(2-hydroxyphenyl)02,4-dioxobutanoate (90 mg, 0.40 mmol, 1 equiv) and (E)-N-(4-fluorobenzyl)-1-(3-phenoxyphenyl)methanimine (122 mg, 0.40 mmol, 1 equiv) were added in 5 mL glacial acetic acid to a 10 mL round bottom flask. The reaction was heated to reflux for 30 minutes while stirring. The mixture was cooled, slowly neutralized with cold methanol, and evaporated in vacuo to yield a crude oil. This oil was redissolved in dichloromethane, washed with saturated sodium chloride, dried over anhydrous sodium sulfate, filtered, and evaporated in vacuo. The compound was purified using flash column chromatography with a gradient of 100% hexanes to 50% hexanes/50% ethyl acetate in to yield KH-1-2 as an off-white solid (25% yield, 99% pure)¹H NMR (400 MHz, chloroform-d) δ 8.16 (dd, J=8.0, 1.7 Hz, 1H), 7.78-7.68 (m, 2H), 7.46 (ddd, J=8.2, 6.8, 1.4 Hz, 1H), 7.34 (dt, J=9.4, 7.7 Hz, 3H), 7.18-7.11 (m, 3H), 7.03-6.98 (m, 4H), 6.97 (t, J=1.4 Hz, 1H), 6.95 (d, J=2.0 Hz, 1H), 6.88 (t, J=2.0 Hz, 1H), 5.23 (d, J=14.4 Hz, 2H), 3.83 (d, J=14.9 Hz, 1H); 13C NMR (101 MHz, chloroform-d) δ 173.2, 162.5 (d, J=247.2 Hz), 162.0, 161.3, 158.2, 158.1, 156.5, 154.4, 154.1, 134.8, 134.6, 131.7, 131.7, 130.6, 130.4, 130.3, 129.9, 127.7, 126.1, 125.5, 123.8, 122.2, 119.2, 119.1, 119.1, 118.0, 116.0, 115.8, 59.2, 43.7; IR vmax (cm⁻¹) 3066, 3047, 2916, 2857, 1711, 1652, 752; HRMS m/z calculated for C₃₀H₂₁FNO₄ [M+H]⁺: 478.1449, found 478.1437.

### Cell-Based Infection Screening Assay

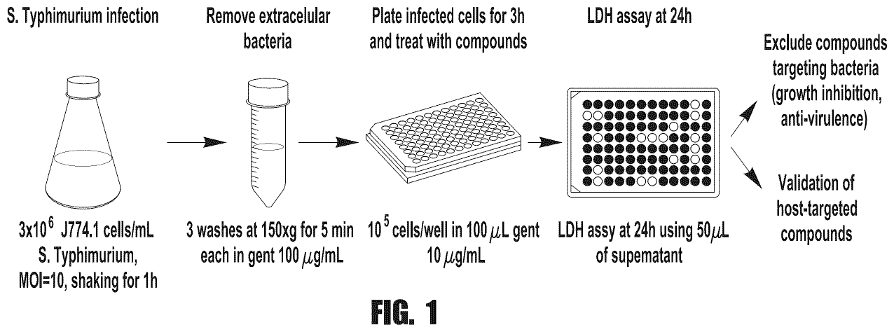

The strategy to identify host-targeted compounds is outlined in FIG. 1. Briefly, J774.1 macrophages in suspension at 3×10⁶ cells/ml were infected with S. typhimurium at a multiplicity of infection (MOI) of 10 for 1 hour with orbital shaking at 80 rpm. The extracellular bacteria were eliminated and removed by the addition of 100 μg/ml gentamicin to the culture medium for 30 min then washed three times using DMEM by centrifugation at 160×g for 10 min each. The infected macrophages were resuspended in a medium containing 10 μg/ml gentamicin, seeded onto 96-well plates at 10⁵ cells/well in 150 μl medium, and allowed to adhere to the well for three hours. The infected cells in each well were then treated with 25 μM of each compound from the ATP mimetic library using a multichannel pipette. The positive control well was treated with 170 μg/ml gentamicin which exhibits lethality to intracellular bacteria with prolonged incubation. After 24 hours of incubation, an LDH assay (Roche Applied Science, Indianapolis, IN) was performed using 50 μl of supernatant from each well. Absorbance at 570 nm was determined via a plate reader Spectra Max M3. The compounds that reduced LDH release from Salmonella-infected macrophages compared with that from untreated wells were selected for subsequent screening and tested for a lack of direct killing of Salmonella.

### Broth Antibacterial Assay

Overnight Salmonella cultures were sub-cultured (1:50) in fresh LB broth containing various concentrations of the compound or in combination with a sub-optimal dose of ciprofloxacin and incubated at 37° C. with aeration. An equivalent amount of DMSO was added in a control group. The bacterial growth was monitored at an optical density at 600 nm (OD600) at the indicated time points using a plate reader Spectra Max M3.

### Neutrophil Reactive Oxygen Species Production Assay

PLB-985 cells that were cultured in RPMI supplemented with 10% FBS and penicillin-streptomycin (100 μg/ml each) were differentiated to a neutrophil-like phenotype by 6-day incubation in RPMI supplemented with 0.5% N,N-dimethylformamide, 0.5% FBS, 1% Nutridoma-SP (Roche; Mannheim, Germany), 2 μmM L-glutamine, and 1× penicillin/streptomycin. Media was replaced on day 3. On day 6 after differentiation, the medium was removed and replaced with 100 μl fresh medium containing different concentrations of KH-1-2. Cells in the positive control group were stimulated with 400 ng PMA/ml. Cells in the negative control group were treated with an equivalent concentration of DMSO. All groups were supplied with luminol (final concentration of 500 μM) and reactive oxygen species (ROS) production at 37° C. was monitored in triplicate by luminol-dependent chemiluminescence measured every 2 minutes for 1 hour using a Spectra Max M3 plate reader.

### Bacterial Cultures and Analysis of Bacterial Growth in Macrophages

Analysis of the inhibitory effects of the compounds on bacterial growth in macrophages was performed as described previously. Hoang et al., International Journal of Pharmaceutics, 2014. 477(1-2): p. 334-343. Briefly, overnight cultures of S. typhimurium or S. typhi were sub-cultured (1:50) in fresh LB broth and incubated for 4 h at 37° C. with aeration. Bacteria were then collected by centrifugation at 3,000×g for 10 min and suspended in phosphate-buffered saline (PBS) to an optical density of 0.6 at 600 nm (5×10⁸ CFU/ml). J774.1 or THP-1 cells were infected with Salmonella strains at an MOI of 10 in the presence of 10% serum in DMEM and RPMI 1640, respectively (Gibco-Life Technologies). One hour after infection, extracellular bacteria were removed by the addition of 100 μg/ml gentamicin to the culture medium for 30 min, and the cell layer was thoroughly washed three times with pre-warmed PBS at 37° C. The infected cells were then treated with different concentrations of each compound or in combination with a sub-optimal dose of ciprofloxacin in a fresh culture medium containing 10% FBS and 10 μg/ml gentamicin that inhibited potential re-infection by extracellular bacteria. At 24 hrs post-treatment, the infected cells were lysed with 0.1% Triton X-100 (Calbiochem, San Diego, CA) in PBS for 10 min. The cell lysates were then serially diluted with PBS and drip plated on LB agar plates. The intracellular bacterial loads were determined by enumerating CFU after 24 h incubation at 37° C.

### Mice

Pathogen-free 7- to 8-week-old female BALB/c mice were purchased from Jackson Labs. Mice were provided food and water ad libitum, divided into groups in sterile micro-isolator cages, and allowed to acclimate for 2 to 3 days before the experiments. All experimental procedures were performed in strict accordance with guidelines established by Nationwide Children's Hospital Institutional Animal Care and Use Committees (IACUCs), and all efforts were made to minimize animal discomfort.

### Toxicity Study of KH-1-2 In Vivo

To examine the toxicity of KH-1-2, mice were intraperitoneally (i.p.) given KH-1-2 that was dissolved in 200 μl of polyethylene glycol 400 (PEG 400)-0.9% saline-ethanol (50:35:15) at 1 mg and 10 mg/kg of body weight per day for 12 consecutive days. Mice in the control groups received 200 μl of PEG-saline-ethanol. The experimental animals were observed daily throughout the study for clinical signs, and mortality. At the end of the experiment, mice were sacrificed, and liver, spleen, and kidney were collected, fixed in 4% paraformaldehyde for 72 hrs, processed and stained with hematoxylin and eosin (H&E) for histopathologic evaluation, which was performed at the Morphology Core at Nationwide Children's Hospital.

### Mouse Model of Typhoid Fever and Evaluation of Protective Efficacy of KH-1-2

S. typhi is a human-restricted pathogen and is unable to colonize in mice. However, S. typhimurium causes a typhoid fever-like disease in mice and is widely used as a model to study human typhoid fever. Johnson, R., E. Mylona, and G. Frankel, Cell Microbiol, 2018. 20(9): p. e12939. Briefly, an overnight culture of a ciprofloxacin-resistant S. typhimurium strain was sub-cultured (1:50) in fresh LB broth and incubated for 6 h at 37° C. with aeration. Bacteria were then collected by centrifugation and suspended in PBS to an optical density of 0.6 at 600 nm (5×10⁸ CFU/ml), then diluted to a concentration of 5×10⁶ CFU/ml in PBS. Mice were infected with the ciprofloxacin-resistant S. typhimurium at a lethal dose of 10⁶ CFU per mouse in 200 μl PBS via the i.p. route.

After determining the maximum tolerable dose of KH-1-2 by the i.p. route (10 mg/kg of body weight per day), the protective efficacy of KH-1-2 as a treatment for typhoid fever was evaluated. Mice (4 or 5 mice per group) were infected at day 0 with a lethal dose of the ciprofloxacin-resistant S. typhimurium isolate as described above. One day post-infection, the infected mice were given KH-1-2 prepared in 200 μl PBS at 0.05, 0.1, and 0.25 mg/kg body weight per day via i.p. delivery for 14 consecutive days.

### Statistical Analysis

Data are presented as mean±standard deviation (SD). p-values were calculated using one-way ANOVA for multiple comparisons and adjusted with Bonferroni's correction or using a non-paired Student's t-test, where two group means were compared; *p<0.05; **p<0.01; ***p<0.001; NS, not significant. For the animal experiments, p-values were calculated using log-rank (Mantel-Cox) test with respect to the PBS control. Statistical analysis was performed using GraphPad Prism 9.

### Results

The Cell-Based Assay Screen for Compounds that Protect S. typhimurium-Infected Macrophages from Death

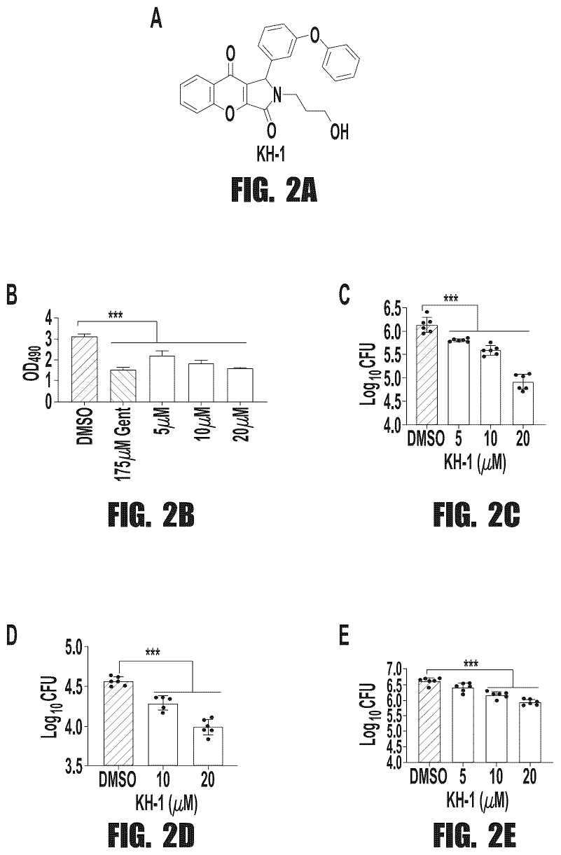

We developed a cell-based assay to screen 3000 compounds of an ATP-mimetic library purchased from ChemBridge using J774.1 macrophages for those that protect S. typhimurium-infected macrophages from death. The viability of Salmonella-infected J774.1 cells was determined by measuring LDH release from the infected cells. The monolayer of infected macrophages was prepared in 96-well plates. Test compounds were added at the final concentration of 25 μM and remained for the duration of the experiment. At 24 hours post-treatment, 50 μl of supernatant from each well was collected and screened for compounds that reduced LDH release in comparison to the control untreated S. typhimurium-infected cells (FIG. 1). The initial screening identified eight hits and subsequent re-screening using a 24-well plate format narrowed the list to three compounds. The most promising compound was 2-(3-hydroxypropyl)-1-(3-phenoxyphenyl)-1,2-dihydrochromeno [2,3-c] pyrrole-3,9-dione that we named KH-1 (FIG. 2A), which reduced LDH release from S. typhimurium-infected macrophages in a dose-dependent manner (FIG. 2B). To examine whether the reduction in LDH release was due to the limited growth of the bacteria intracellularly, the infected cells were treated with various concentrations of KH-1, and the intracellular bacterial load was determined at 24 hours post-treatment. As shown in FIG. 2C and D, KH-1 reduced intracellular bacterial growth of both antibiotic sensitive and ciprofloxacin-resistant strains respectively in a dose-dependent manner. The intracellular antibacterial effects of KH-1 are not dependent on host cell species since the compound also limits S. typhimurium growth in THP-1 human macrophages (FIG. 2E).

KH-1 is not Antibacterial in Standard Medium and does not Affect Salmonella Entry into Macrophages

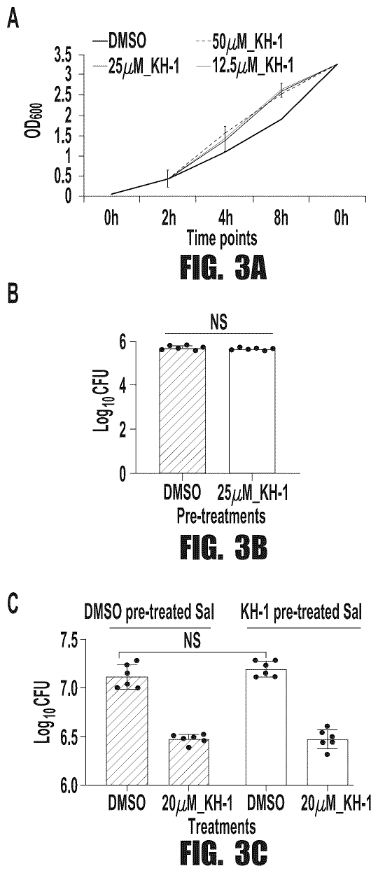

To test whether KH-1 has direct inhibitory effects on bacteria in broth, we examined bacterial growth in the presence of various concentrations of KH-1. As shown in FIG. 3A, even with a concentration of 50 μM, which is 10-fold above the lowest concentration showing an effect on LDH release, KH-1 does not exert direct inhibitory effects on the growth of S. typhimurium in vitro. It is also possible that the compound may negatively affect the entry of Salmonella into macrophages. To test this hypothesis, we pre-cultured S. typhimurium in LB containing 20 μM KH-1 and then compared their infectivity with bacteria that were pre-cultured in LB without KH-1. As shown in FIG. 3B, there is no significant difference in infectivity between bacteria pre-cultured in KH-1 and those bacteria pre-cultured in LB alone. In addition, there is no difference in the intracellular growth of pre-cultured bacteria in KH-1 and LB alone at 24 hours post-infection (FIG. 3C). These data suggested that pre-exposing Salmonella to KH-1 does not alter the invasive ability and intracellular growth of the bacteria. Since KH-1 does not directly inhibit the growth or ability of the bacteria to invade macrophages but does reduce bacterial growth intracellularly, we hypothesized that the compound modulates the host cell to limit intracellular bacterial survival.

### KH-1 Sensitizes Bacteria to Ciprofloxacin in the Infected Macrophages but not in a Standard Medium

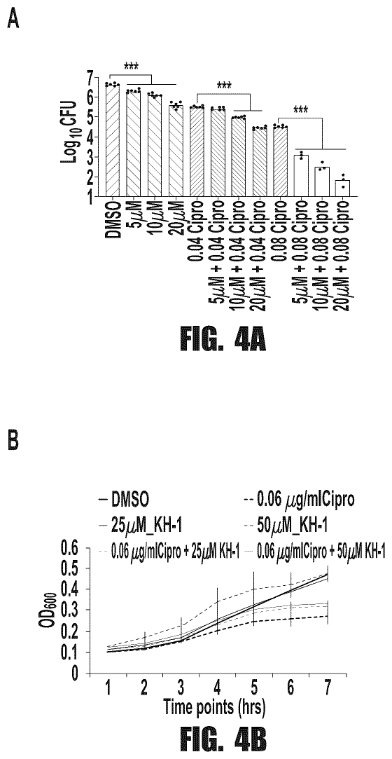

We next addressed whether the lead compound could be used in combination with ciprofloxacin, a common antibiotic used to treat typhoid fever. To this end, we treated Salmonella-infected cells with various concentrations of KH-1 and a sub-optimal dose of ciprofloxacin (determined before the experiments) and determined the intracellular bacterial load at 24 hours post-treatment. As shown in FIG. 4A, KH-1 treatment with sub-optimal doses of ciprofloxacin consistently reduced intracellular bacterial growth to a greater extent than KH-1 alone. Importantly, KH-1 treatment did not sensitize the bacteria to ciprofloxacin in a standard bacterial culture medium (FIG. 4B). These data further demonstrate the potential of KH-1 in treating Salmonella systemic infection.

### Screening for More Potent KH-1 Analogs Identified KH-1-2

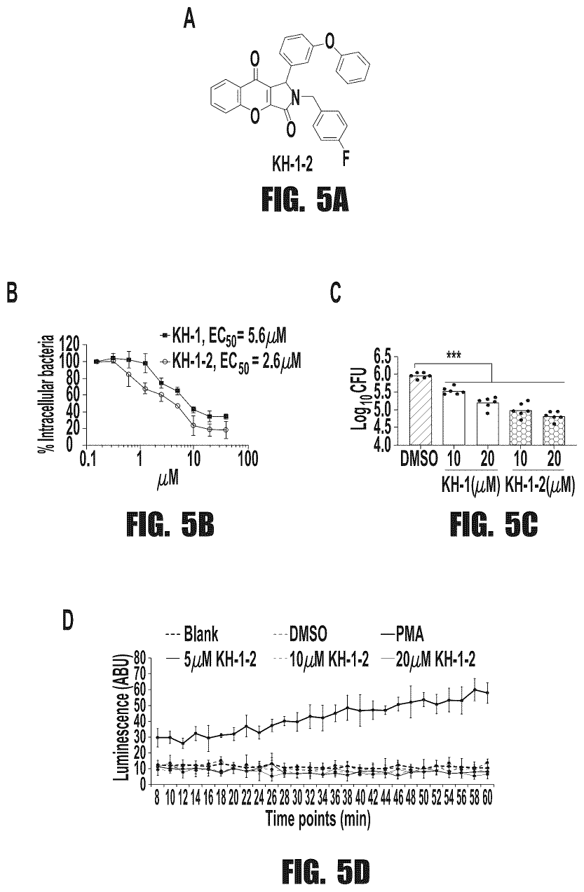

To improve the activity of KH-1, we performed an online search for potential analogs and experimentally examined identified compounds for toxicity and Salmonella intramacrophage survival. From screening four potential analogs, we identified a compound (2-(4-fluorobenzyl)-1-(3-phenoxyphenyl)-1,2-dihydrochromeno[2,3-c] pyrrole-3,9-dione that we named KH-1-2 (FIG. 5A) that is more effective at reducing the intracellular growth of S. typhi (FIG. 5B) and S. typhimurium (FIG. 5C) than KH-1. As shown in FIG. 5C, KH-1-2 has a half maximal effective concentration (EC₅₀) of 2.6 μM in comparison with the EC₅₀ of 5.6 μM of KH-1 on S. typhimurium. The other three compounds tested were less effective than KH-1.

Induction of ROS production is a potent mechanism for targeting infection, but prolonged excessive intracellular ROS production can lead to activation of cell death. To determine if KH-1-2 addition activated ROS, PLB-985 neutrophil-like cells were treated with various concentrations of KH-1-2. These data demonstrate that the host-mediated anti-Salmonella activity of KH-1-2 does not involve in induction of reactive oxygen species (ROS) production (FIG. 5D). KH-1-2 alone is also not toxic to macrophages at the effective concentrations as measured by LDH release.

### Evaluation of Toxicity in Mice Following KH-1-2 Administration

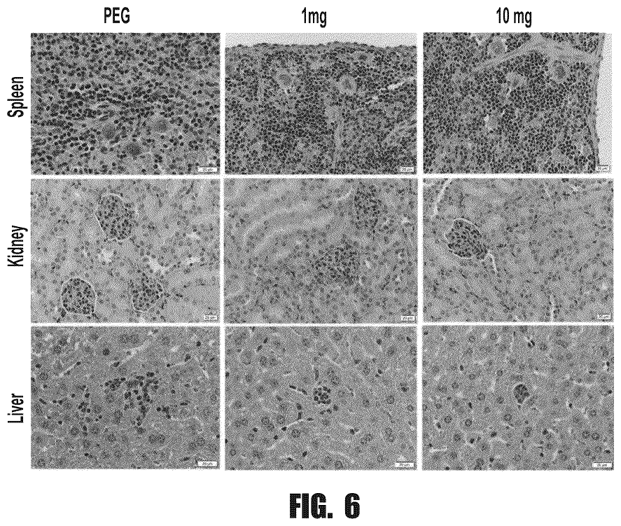

To examine the in vivo toxicity of KH-1-2, uninfected mice were given KH-1-2 intraperitoneally in 200 μl of polyethylene glycol 400 (PEG 400)-0.9% saline-ethanol (50:35:15) at 1 and 10 mg/kg body weight daily for 12 consecutive days. Mice in the control group were treated with an equal amount of diluent. Mice were monitored for clinical signs and at day 13, the liver, spleen, and kidney were collected for histopathologic evaluation. KH-1-2 treatment resulted in no adverse clinical signs and no dose-related macroscopic or microscopic findings upon histologic evaluation of the liver, spleen, and kidney (FIG. 6). No increase in immune cell migration or significant apoptosis in these organs was observed in treated mice. For the histopathology study of the liver, KH-1-2 treatment at 1 mg/kg resulted in no adverse effects, however, treatment at 10 mg/kg resulted in minimal alterations including increased infiltration of lymphocytic cells and neutrophils. Overall, these data indicate that the i.p. route of delivery of 1 and 10 mg/kg of KH-1-2 is well-tolerated in mice.

KH-1-2 Treatment of Mice Infected with an Antibiotic-Resistant S. typhimurium Isolate Protects Against Lethality

We next sought to examine the effectiveness of delivering KH-1-2 for controlling a ciprofloxacin-resistant S. typhimurium isolate in a mouse model of typhoid fever. Mice were challenged with a lethal dose of bacteria via oral gavage and the infected mice were i.p. treated with KH-1-2 prepared in 200 μl PBS at 0.05, 0.1, and 0.25 mg/kg body weight/day for 14 consecutive days. These concentrations were driven by experiments in mice with the parent compound KH-1, which showed protection or increased time to death at a ˜10-fold higher range of the compound. As expected, all mice in the control group were moribund before or at day 12 post-infection. We achieved significantly greater survival with KH-1-2 treatment groups than with the control untreated group (p<0.05), with all doses providing some protection from death (FIG. 7). The dose of 0.1 mg/kg body weight/day was most effective, showing 60% survival.

### Discussion

In this study, we attempted to identify new host-targeted therapeutic candidates for the treatment of typhoid fever caused by both antibiotic-sensitive and resistant bacterial strains. The treatment of typhoid fever is confined to a few antibiotic options, including ciprofloxacin, azithromycin, and ceftriaxone. However, recent epidemiological studies reported the development of resistance to these drugs, making treatment failure too frequent. Raveendran, R., et al., Indian J Med Microbiol, 2008. 26(1): p. 50-3. There are several typhoid fever vaccines available for adults and children, but they provide variable protection and can cause side effects. New antimicrobial discovery has been lacking and the incidence of Salmonella infections and the rapid emergence of multidrug-resistant S. typhi has increased, highlighting the worldwide threat of this pathogen and the urgent need for new approaches for therapeutic intervention.

Modulation of the host immune system as well as targeting host factors employed by pathogenic microbes for infection and survival are emerging approaches to control intracellular pathogens. Finlay, B. B. and R. E. Hancock, Nat Rev Microbiol, 2004. 2(6): p. 497-504. Salmonella spp have evolved multiple mechanisms to counteract and exploit host immune pathways to evade killing, subsequently inducing cell death and the induction of the inflammatory response to promote disease. Boise, L. H. and C. M. Collins, Trends in Microbiology, 2001. 9(2): p. 64-67. Thus, drugs that prevent Salmonella-induced cell death could be used as a therapy to control typhoid fever.

Host cell death assay has been developed for the screening of or evaluation of active molecules against microbial pathogens. Cruz, D. J., et al., PLoS Negl Trop Dis, 2013. 7(10): p. e2471. Here we report the development of a simple, cell-based assay using lactate hydrogenase (LDH) as a readout to identify compounds that protect Salmonella infected macrophages from death. An initial screening of a 3000 compound ATP-mimetic library, a subset of the ChemBridge ATP mimetic (kinase inhibitor) library identified eight active compounds that inhibited Salmonella-mediated cell death. Subsequent screening of the eight compounds focused on KH-1 (FIG. 2A) that reduced LDH release and inhibited Salmonella growth inside infected cells (FIGS. 2C, D, and E)) with an EC₅₀ in the single-digit micromolar range (FIG. 5B).

Pre-exposure of Salmonella to KH-1 does not alter bacterial invasion (FIG. 3B) or proliferation inside macrophages (FIG. 3C) that, combined with the observation that the compound does not directly inhibit bacterial growth in broth (FIG. 3A), suggests the lack of direct bacterial target for KH-1. The compound does not affect the host cell proliferation (Sup. FIG. 4) nor inhibit bacterial invasion (Sup. FIG. 5). The compound also acts in an additive and/or synergistic mode with a suboptimal dose of ciprofloxacin in the infected macrophage (FIG. 4A) but not in the broth (FIG. 4B). Together, these data indicate a host-directed mechanism of the compound is responsible for its anti-Salmonella activity.

Our study showed that the compound is effective in both mouse (FIGS. 2C and D) and human macrophages (FIG. 2E), suggesting that it targets a common pathway in humans and mice. Additionally, the host-targeted activity of the compound was confirmed against an antibiotic-resistant clinical isolate (FIG. 2D). With the increase in MDR infections coupled with the lack of investment in novel antibacterials by pharmaceutical companies, combinatorial treatment regimens using multiple complementary approaches including host-targeted therapy in combination with conventional antibiotics are likely going to be indispensable to controlling and eradicating Salmonella infections. Our study showed that when combined with sub-optimal doses of antibiotics, KH-1 further inhibited intracellular bacterial growth inside the macrophages (FIG. 4A), suggesting the continued study of a dual treatment regimen of the compound with antibiotics to control typhoid fever.

We examined the toxicity and therapeutic efficacy of KH-1-2 by using a mouse model. No adverse clinical signs were observed from KH-1-2 treated mice at a concentration up to 10 mg/kg/day for 10 days. Histopathological studies of liver, spleen, and kidney from KH-1-2 treated mice showed a small increase in infiltration of immune cells in the liver, but not in the other two organs compared to that in control mice (FIG. 6). Thus, KH-1-2 does not appear to have significant toxicities. A more comprehensive evaluation of the potential adverse effects of KH-1-2 in vivo is planned for future experiments. We then used KH-1-2 to treat typhoid fever in a mouse model using a ciprofloxacin-resistant Salmonella strain. KH-1-2 was delivered from day 1 to 12 post-infection via i.p. route. Mice in KH-1-2 treated groups (0.05 mg/kg-0.25 mg/kg) were protected from lethal infection (FIG. 7). These effective doses were much lower than the doses showing no or minimal toxicity dose (1-10 mg/kg), suggesting a potential wide therapeutic window of the compound. However, we observed that a higher dose of the compound provided less protective efficacy, perhaps due to partial agonism/antagonism activity of the target or off-target effects. More comprehensive optimization of the effective dose and delivery route is under investigation.

Reactive oxygen species (ROS) produced by the host plays an important role in controlling microbial infection, including in those infected by Salmonella. Herb, M. and M. Schramm, Functions of ROS in Macrophages and Antimicrobial Immunity. Antioxidants (Basel), 2021. 10(2). Paradoxically, dysregulation or excessive production of ROS induces damage to the host and promotes particular infections. Paiva, C. N. and M. T. Bozza, Antioxidants & Redox Signaling, 2014. 20(6): p. 1000-1037. Our data showed that KH-1-2 does not induce ROS production (FIG. 5D). The cellular mechanisms that are targeted by KH-1-2 that result in the reduction of Salmonella growth inside the macrophages and increase survival of infected mice are unknown but are actively being elucidated.

In summary, the work presented describes the application of a cell-based throughput assay system using an LDH assay approach to screen a chemical library for host-targeted compounds active against Salmonella. These studies provide the support that KH-1-2 may be a promising lead compound for the development of new host-targeted therapeutic agents to control typhoid fever caused by both antibiotic sensitive and resistant Salmonella strains.

### Example 2: KH-2 Effect on Salmonella Infection

KH-2 was tested for its ability to inhibit Salmonella infection. Overnight cultures of S. typhimurium were prepared for infection of J774.1 cells by sub-culture (1:50) in fresh Luria-Bertani (LB) broth and incubated for 4 h at 37° C. Bacteria were then collected by centrifugation at 3000×g for 10 min and suspended in phosphate buffer saline (PBS) to an optical density of 0.6 at 600 nm which was equivalent to 10⁹ CFU/mL. J774.1 cells were seeded in 24-well plate at a density of 2×10⁵ cells/well for overnight and then were infected with S. typhimurium at a multiplicity of infection (MOI) of 10 in the presence of 10% fetal bovine serum (FBS) in RPMI 1640 (Gibco-Life Technologies). Two hours after infection, extra-cellular bacteria were removed by addition of 100 μg/mL of gentamicin to the culture medium for 1 h and then the cell layer was thoroughly washed three times with pre-warmed RPMI 1640. The infected J774.1 cells were then treated with KH-2. Gentamicin was also added at 10 mg mL 1 to eliminate potential re-infection by extracellular bacteria. After 24 h, the supernatant was collected and subjected to lactate dehydrogenase (LDH) assay by following manufacture's instruction. FIG. 9 provides a graph showing KH-2 (5 μM, 10 μM, and 20 μM) reduced lactate dehydrogenase (LDH) release from Salmonella infected J774.1 macrophages, which is a measure of cell death.

### Example 3: KH-2 Inhibit the Growth of S. typhimurium in J774.1 Macrophages

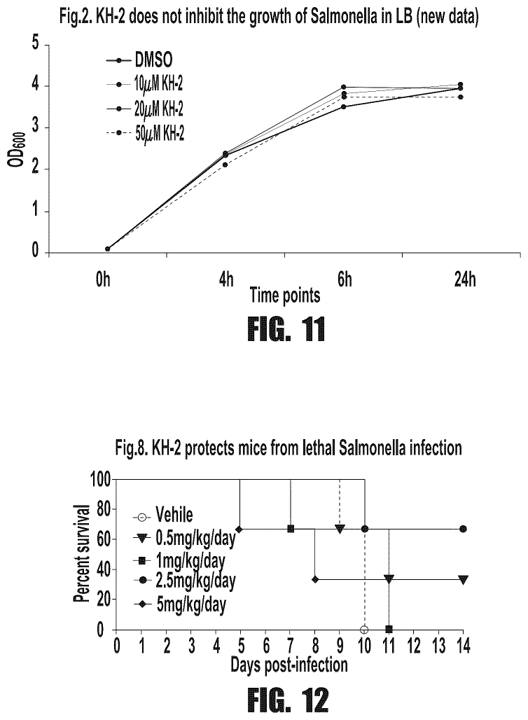

The J774.1 cells were infected with S. typhimurium and treated with KH-2. After 24 hours, the infected cells were lysed with 0.1% Triton X-100 in phosphate saline buffer (PBS) for 15 min. The cell lysates were then serially diluted with PBS and spread on LB agar plates. The surviving intracellular bacteria were determined by enumerating CFU after 24 h incubation at 37° C. FIG. 10 provides a graph showing KH-2 (5 μM, 10 μM, and 20 μM) inhibits the growth of Salmonella in J774.1 macrophages, with increasing doses of KH-2 resulting in increased inhibition.

### Example 4: KH-2 does not Inhibit the Growth of S. typhimurium in LB Broth

Overnight cultures of S. typhimurium were sub-cultured (1:100) in fresh Luria-Bertani (LB) broth in presence of different concentration of KH-2 and incubated at 37° C. The vehicle control (DMSO) groups were included. The experiments were performed in 96-well plates. The bacterial growth was monitored at different time-points by measuring optical density at 600 nm (OD₆₀₀). FIG. 11 provides a graph showing that KH-2 (10 μM, 20 μM, and 50 μM) does not inhibit the growth of Salmonella in Luria-Bertani (LB) bacterial growth media, showing that the effect is only present for intracellular pathogens.

### Example 5: KH-2 Protect Mice from Lethal S. typhimurium Infection

Pathogen-free 6- to 8-week-old female BALB/c mice were purchased from Harlan Sprague. Mice (3 mice/group, except the vehicle control group) were provided food and water ad libitum in sterile microisolator cages and allowed to acclimatize for 3 days before challenge. Mice were orally infected with 10⁶ bacteria/mouse (day 0) in 200 μl PBS. The infected mice were intraperitoneally given KH-2 at different doses (5 mg, 2.5 mg, 1 mg, and 0.5 mg/kg body weight/day) in 200 μl PBS from day 1 to day 12 post infection. Vehicle (DMSO) was added as the control group. The mice were monitored for survival until day 14 post infection. FIG. 12 provides a graph showing that KH-2 (0.5, 1.0, 2.5, and 5 mg/kg/day) protects mice from lethal Salmonella infection, with increasing doses showing an increased effect. A mouse model of typhoid fever was used to assess the efficacy of KH-2 in increasing the survival rate of infected mice.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating or preventing infection by an intracellular pathogen in a subject, comprising administering to the subject a therapeutically effective amount of a compound according to Formula I: 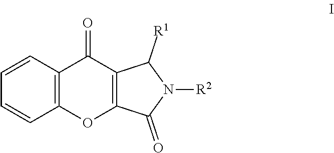
wherein R¹ is a C₆-C₁₂ alkyl, phenyl, or heteroaryl group, and R² is C₁-C₆ alkyl group or a phenyl group, wherein one or more of the hydrogens of the phenyl group are optionally substituted with a methyl, halogen, —OMe, or —OPh moiety; or Formula II: 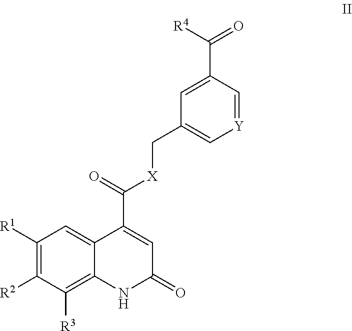
wherein R¹, R², and R³ are either a hydrogen or halogen, wherein R⁴ is —NHMe or —OMe, X is O or NH, and Y is C or N; or a pharmaceutically acceptable salt thereof.

2. The method of claim 1, wherein the compound is according to Formula I.

3. The method of claim 2, wherein R¹ is diphenyl ether.

4. The method of claim 3, wherein R² is n-propanol.

5. The method of claim 3, wherein R² a phenyl group, wherein one or more of the hydrogens of the phenyl group are substituted with a halogen.

6. The method of claim 1, wherein the compound is according to Formula II.

7. The method of claim 6, wherein R¹-R³ are —H, R⁴ is —OMe, X is O, and Y is C.

8. The method of claim 1, wherein the composition is administered to prevent infection.

9. The method of claim 1, wherein the composition is administered to treat infection.

10. The method of claim 1, wherein the intracellular pathogen is a Francisella species.

11. The method of claim 10, wherein the Francisella species is Francisella tularensis.

12. The method of claim 1, wherein the intracellular pathogen is a Salmonella species.

13. The method of claim 12, wherein the Salmonella species is Salmonella typhimurium.

14. The method of claim 12, wherein the Salmonella species is Salmonella typhi.

15. The method of claim 1, wherein intracellular pathogen inhabits macrophage cells.

16. The method of claim 1, wherein intracellular pathogen is antibiotic resistant.

17. The method of claim 1, wherein the composition is administered with a pharmaceutically acceptable carrier.

18. A method of treating or preventing bacterial inflammation in a subject, comprising administering to the subject a therapeutically effective amount of a composition according to Formula I: 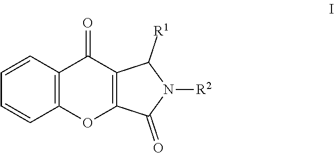
wherein R¹ is a C₆-C₁₂ alkyl, phenyl, or heteroaryl group, and R² is C₁-C₆ alkyl group or a phenyl group, wherein one or more of the hydrogens of the phenyl group are optionally substituted with a methyl, halogen, —OMe, or —OPh moiety; or Formula II: 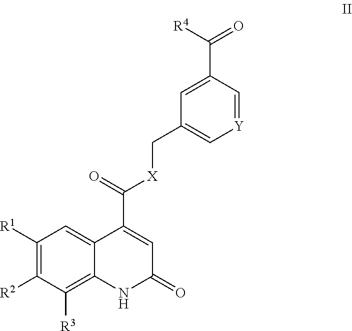
wherein R¹, R², and R³ are either a hydrogen or halogen, wherein R⁴ is —NHMe or —OMe, X is O or NH, and Y is C or N; or a pharmaceutically acceptable salt thereof.

19. The method of claim 18, wherein the compound is according to Formula I.

20. The method of claim 18, wherein the compound is according to Formula II.

21. The method of claim 18, wherein the inflammation is systemic LPS inflammation.

22. A composition comprising a compound to according to Formula I: 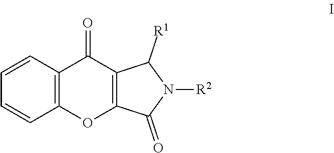
wherein R¹ is a C₆-C₁₂ alkyl, phenyl, or heteroaryl group, and R² is a phenyl group, wherein one or more of the hydrogens of the phenyl group are optionally substituted with a methyl, halogen, —OMe, or —OPh moiety, or a pharmaceutically acceptable salt thereof.

23. The composition of claim 22, wherein R¹ is diphenyl ether.

24. The composition of claim 22, wherein one or more of the hydrogens of the phenyl group is substituted with a halogen.

25. The composition of claim 22, wherein the composition further comprises a pharmaceutically acceptable carrier.

26. A composition comprising a compound according to Formula II: 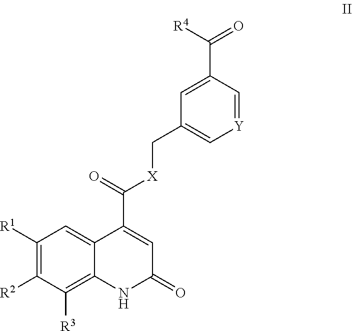
wherein R¹, R², and R³ are either a hydrogen or halogen, wherein R⁴ is —NHMe or —OMe, X is O or NH, and Y is C or N; or a pharmaceutically acceptable salt thereof.

27. The composition of claim 26, wherein one or more of R¹, R², and R³ is a halogen.

28. The composition of claim 26, wherein X is NH.

29. The composition of claim 26, wherein R⁴ is —NHMe.

30. The composition of claim 26, wherein the composition further comprises a pharmaceutically acceptable carrier.
